(19) United States
(12) Patent Application Publication
(10) Pub. No.: US 2016/0245814 A1
(43) Pub. Date: Aug. 25, 2016

(54) BIOMARKERS FOR KIDNEY CANCER AND METHODS USING THE SAME

(71) Applicant: METABOLON, INC., Durham, NC (US)

(72) Inventors: Bruce NERI, Cary, NC (US); Steven M. STIRDIVANT, Cary, NC (US)

(21) Appl. No.: 15/026,341

(22) PCT Filed: Sep. 26, 2014
(86) PCT No.: PCT/US14/57650

§ 371 Date: Mar. 31, 2016

Related U.S. Application Data
(60) Provisional application No. 61/886,264, filed on Oct. 3, 2013.

(51) Int. Cl. G01N 33/574

(52) U.S. Cl. CPC G01N 33/57438; G01N 2800/7028; G01N 2800/56

(57) ABSTRACT

Methods for identifying and evaluating biochemical entities useful as biomarkers for kidney cancer are described. Suites of small molecule entities as biomarkers for clear cell papillary kidney cancer are also described.

### CROSS-REFERENCE TO RELATED APPLICATIONS

This application claims the benefit of U.S. Provisional Patent Application No. 61/886,264, filed Oct. 3, 2013, the entire contents of which are hereby incorporated herein by reference.

### FIELD

The invention generally relates to biomarkers for clear cell papillary kidney cancer and methods based on the same biomarkers.

### BACKGROUND

In the US, 275,000 patients each year are screened for kidney cancer, and 55,000 are diagnosed with renal cell carcinoma (RCC) (American Cancer Society Facts and Figures 2010). RCC is the most common form of kidney cancer, accounting for approximately 80% of the total. The incidence of RCC is steadily increasing, and in the US increased by approximately 2% per year in the past two decades (Ries L A G, et al., eds. SEER Cancer Statistics Review, 1975-2003. Bethesda, Md.: National Cancer Institute; 2006). Because RCC is one of the deadliest cancers and does not respond to traditional chemotherapy drugs, many new targeted agents are being developed specifically to treat RCC.

70% of newly diagnosed patients are diagnosed in the early stages (T1 and T2). Early stage RCC is treated by partial or total nephrectomy; this is surgery with curative intent. When RCC tumors are surgically removed at an early stage, the 5 year survival rate is 90% for stage 1 and 51% for stage 2, yet 70% of RCC patients develop metastasis during the course of their disease.

Clear cell papillary renal cell carcinoma (CCPRCC) is a recently described subtype of RCC that appears to be morphologically and genetically distinct from clear cell RCC and papillary RCC. This subtype presents less frequently than either clear cell RCC or papillary RCC, presents with low pathological stage, and appears to be clinically indolent. Further, CCPRCC tumors are associated with a lower metastatic potential and a better prognosis than clear cell RCC. As CCPRCC is a new subtype of RCC, the morphological differences from other types of RCC may not be readily apparent. Additional analysis, such as immunohistochemistry, genetic analysis or biochemical analysis (small molecule biomarkers), may be needed to confirm classification. Given that CCPRCC does not exhibit the aggressive pathological features often associated with clear cell RCC and papillary RCC, there is clinical benefit in distinguishing CCPRCC from other subtypes of RCC. The ability to distinguish CCPRCC from the other RCC subtypes will allow the physician to prescribe therapies specific for the type of RCC.

### SUMMARY

In one aspect, the present invention provides a method of diagnosing whether a subject has CCPRCC, comprising analyzing a biological sample from a subject to determine the level(s) of one or more biomarkers for CCPRCC in the sample, where the one or more biomarkers are selected from sorbitol, fructose, sorbitol 6-phosphate, myristate, palmitate and stearate and comparing the level(s) of the one or more biomarkers in the sample to CCPRCC-positive and/or CCPRCC-negative reference levels of the one or more biomarkers in order to diagnose whether the subject has CCPRCC.

In a further aspect, the invention provides a method of distinguishing CCPRCC from other subtypes of kidney cancer (e.g., clear cell RCC, papillary RCC, chromophobe RCC), comprising analyzing a biological sample from a subject to determine the level(s) of one or more biomarkers for CCPRCC in the sample where the one or more biomarkers are selected from sorbitol, fructose, sorbitol 6-phosphate, myristate, palmitate and stearate and comparing the level(s) of the one or more biomarkers in the sample to CCPRCC-positive and/or CCPRCC-negative reference levels of the one or more biomarkers in order to distinguish CCPRCC from other subtypes of kidney cancer.

### BRIEF DESCRIPTION OF THE DRAWINGS

FIG. 1. Graphical illustration of box plots of the levels of sorbitol measured in normal biopsy tissue samples or clear cell renal cancer (ccRCC) biopsy tissue samples (circles); the ‘outliers’ (Squares) having very high levels of sorbitol were histologically confirmed to be CCPRCC.

FIG. 2. Graphical illustration of box plots of the levels of the biomarker metabolites measured in normal biopsy tissue samples (left), ccRCC biopsy tissue samples (middle) or CCPRCC (Clear cell/Pap, right).

FIG. 3. Graphical illustration of principal components analysis (PCA) using biopsy tissue from CCPRCC, clear cell RCC (ccRCC) and benign (normal) samples. Ovals illustrate that these metabolic abundance profiles can separate samples into groups.

### DETAILED DESCRIPTION

The present invention relates to biomarkers of CCPRCC, and methods for diagnosis or aiding in diagnosis of CCPRCC. Prior to describing this invention in further detail, however, the following terms will first be defined.

### Definitions:

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, kidney tissue, blood, blood plasma, urine, or cerebral spinal fluid (CSF).

“Subject” means any animal, but is preferably a mammal, such as, for example, a human, monkey, mouse, rabbit or rat.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “CCPRCC-positive reference level” of a biomarker means a level of a biomarker that is indicative of a positive diagnosis of CCPRCC in a subject, and a “CCPRCC-negative reference level” of a biomarker means a level of a biomarker that is indicative of a negative diagnosis of CCPRCC in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, etc.), where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Non-limiting examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Metabolome” means all of the small molecules present in a given organism.

“Kidney cancer” or renal cell carcinoma (RCC) refers to a disease in which cancer develops in the kidney.

“Clear cell papillary renal cell carcinoma” or CCPRCC refers to a subtype of RCC.

“Staging” of kidney cancer refers to an indication of the severity of kidney cancer including tumor size and whether and/or how far the kidney tumor has spread. The tumor stage is a criteria used to select treatment options and to estimate a patient's prognosis. Kidney tumor stages range from T1 (tumor 7 cm or less in size and limited to kidney, least advanced) to T4 (tumor invades beyond Gerota's fascia, most advanced). “Low stage” or “lower stage” kidney cancer refers to kidney cancer tumors, including malignant tumors with a lower potential for recurrence, progression, invasion and/or metastasis (less advanced). Kidney tumors of stage T1 or T2 are considered “low stage”. “High stage” or “higher stage” kidney cancer refers to a kidney cancer tumor in a subject that is more likely to recur and/or progress and/or invade beyond the kidney, including malignant tumors with higher potential for metastasis (more advanced). Kidney tumors of stage T3 or T4 are considered “high stage”.

“Grade” of kidney cancer refers to the appearance and/or structure of kidney cancer cellular nuclei. “Low grade” kidney cancer refers to a cancer with cellular nuclear characteristics more closely resembling normal cellular nuclei. “High grade” kidney cancer refers to a cancer with cellular nuclear characteristics less closely resembling normal cellular nuclei.

“Aggressiveness” of kidney cancer or a cancer-positive small renal mass refers to a combination of the stage, grade, and metastatic potential of a kidney tumor. “More aggressive” kidney cancer refers to tumors of higher stage, grade, and/or metastatic potential. Cancer tumors that are not confined to the kidney are considered to be more aggressive kidney cancer. “Less aggressive” kidney cancer refers to tumors of lower stage, grade, and/or metastatic potential. Cancer tumors that are confined to the kidney are considered to be less aggressive kidney cancer. CCPRCC is generally regarded as a less aggressive subtype of kidney cancer.

“CCPRCC Score” is a measure of the probability that an RCC tumor is a CCPRCC tumor. The CCPRCC Score is based on using the CCPRCC biomarkers in a statistical or mathematical model or algorithm as described herein.

### I. Biomarkers

The CCPRCC biomarkers described herein were discovered using metabolomic profiling techniques. Such metabolomic profiling techniques are described in more detail in the Examples set forth below as well as in U.S. Pat. Nos. 7,005,255, 7,329,489; 7,550,258; 7,550,260; 7,553,616; 7,635,556; 7,682,783; 7,682,784; 7,910,301; 6,947,453; 7,433,787; 7,561,975; 7,884,318, the entire contents of which are hereby incorporated herein by reference.

Generally, metabolic profiles were determined for biological samples from human subjects that were positive for CCPRCC, human samples that were positive for clear cell RCC or samples from human subjects that were cancer negative (non-cancer). The metabolic profile for biological samples positive for CCPRCC was compared to the metabolic profile for biological samples negative for CCPRCC (e.g., clear cell RCC or normal/benign). Those small molecules differentially present, including those small molecules differentially present at a level that is statistically significant, in the metabolic profile of samples positive for CCPRCC as compared to another group (e.g., clear cell RCC or normal) were identified as biomarkers to distinguish those groups.

The biomarkers are discussed in more detail herein. The biomarkers that were discovered correspond with biomarkers for distinguishing samples positive for CCPRCC vs. clear cell RCC and samples positive for CCPRCC vs. Normal.

### II. Methods

A. Diagnosis of CCPRCC

The identification of biomarkers for CCPRCC allows for the diagnosis of (or for aiding in the diagnosis of) CCPRCC in subjects presenting with one or more symptoms consistent with the presence of kidney cancer and includes the initial diagnosis of CCPRCC in a subject not previously identified as having CCPRCC and diagnosis of recurrence of CCPRCC in a subject previously treated for kidney cancer.

A method of diagnosing (or aiding in diagnosing) whether a subject has CCPRCC comprises (1) analyzing a biological sample from a subject to determine the level(s) of one or more biomarkers of CCPRCC in the sample and (2) comparing the level(s) of the one or more biomarkers in the sample to CCPRCC-positive and/or CCPRCC-negative reference levels of the one or more biomarkers in order to diagnose (or aid in the diagnosis of) whether the subject has CCPRCC. The one or more biomarkers that are used are selected from sorbitol, fructose, sorbitol 6-phosphate, myristate, palmitate and stearate and combinations thereof. When such a method is used to aid in the diagnosis of CCPRCC, the results of the method may be used along with other methods (or the results thereof) useful in the clinical determination of whether a subject has CCPRCC.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers selected from the group consisting of sorbitol, fructose, sorbitol 6-phosphate, myristate, palmitate and stearate may be determined in the methods of diagnosing and methods of aiding in diagnosing whether a subject has CCPRCC. For example, one or more of the following biomarkers may be used alone or in combination to diagnose or aid in diagnosing CCPRCC: sorbitol, fructose, sorbitol 6-phosphate, myristate, palmitate and stearate. Additionally, for example, the level(s) of one biomarker, two or more biomarkers, three or more biomarkers, four or more biomarkers, five or more biomarkers, including a combination of all of the biomarkers selected from the group consisting of sorbitol, fructose, sorbitol 6-phosphate, myristate, palmitate and stearate or any fraction thereof, may be determined and used in such methods. Determining levels of combinations of the biomarkers may allow greater sensitivity and specificity in diagnosing CCPRCC and aiding in the diagnosis of CCPRCC. For example, ratios of the levels of certain biomarkers (and non-biomarker compounds) in biological samples may allow greater sensitivity and specificity in diagnosing CCPRCC and aiding in the diagnosis of CCPRCC.

After the level(s) of the one or more biomarkers in the sample are determined, the level(s) are compared to CCPRCC-positive and/or CCPRCC-negative reference levels to aid in diagnosing or to diagnose whether the subject has CCPRCC. Levels of the one or more biomarkers in a sample matching the CCPRCC-positive reference levels (e.g., levels that are the same as the reference levels, substantially the same as the reference levels, above and/or below the minimum and/or maximum of the reference levels, and/or within the range of the reference levels) are indicative of a diagnosis of CCPRCC in the subject. Levels of the one or more biomarkers in a sample matching the CCPRCC-negative reference levels (e.g., levels that are the same as the reference levels, substantially the same as the reference levels, above and/or below the minimum and/or maximum of the reference levels, and/or within the range of the reference levels) are indicative of a diagnosis of no CCPRCC in the subject. In addition, levels of the one or more biomarkers that are differentially present (especially at a level that is statistically significant) in the sample as compared to CCPRCC-negative reference levels are indicative of a diagnosis of CCPRCC in the subject. Levels of the one or more biomarkers that are differentially present (especially at a level that is statistically significant) in the sample as compared to CCPRCC-positive reference levels are indicative of a diagnosis of no CCPRCC in the subject.

The level(s) of the one or more biomarkers may be compared to CCPRCC-positive and/or CCPRCC-negative reference levels using various techniques, including a simple comparison (e.g., a manual comparison) of the level(s) of the one or more biomarkers in the biological sample to CCPRCC-positive and/or CCPRCC-negative reference levels. The level(s) of the one or more biomarkers in the biological sample may also be compared to CCPRCC-positive and/or CCPRCC-negative reference levels using one or more statistical analyses (e.g., t-test, Welch's T-test, Wilcoxon's rank sum test, Random Forest, T-score, Z-score) or using a mathematical model (e.g., algorithm, statistical model).

For example, a mathematical model comprising a single algorithm or multiple algorithms may be used to determine whether a subject has CCPRCC. A mathematical model may also be used to distinguish between CCPRCC and RCC. An exemplary mathematical model may use the measured levels of any number of biomarkers s (for example, 2, 3, 5, etc.) from a subject to determine, using an algorithm or a series of algorithms based on mathematical relationships between the levels of the measured biomarkers, whether a subject has CCPRCC, the probability that a subject with kidney cancer has CCPRCC, etc.

The results of the method may be used along with other methods (or the results thereof) useful in the diagnosis of CCPRCC in a subject.

In one aspect, the biomarkers provided herein can be used to provide a physician with an CCPRCC Score indicating the probability of CCPRCC in a subject. The score is based upon clinically significantly changed reference level(s) for a biomarker and/or combination of biomarkers. The reference level can be derived from an algorithm. The CCPRCC Score can be used to place the subject in a probability range of CCPRCC from low (i.e. normal, no kidney cancer) to high.

Methods for determining a subject's CCPRCC Score may be performed using one or more of the CCPRCC biomarkers selected from the group consisting of sorbitol, fructose, sorbitol 6-phosphate, myristate, palmitate and stearate in a biological sample. The method may comprise comparing the level(s) of the one or more CCPRCC biomarkers in the sample to CCPRCC reference levels of the one or more biomarkers in order to determine the subject's CCPRCC score. The method may employ any number of markers selected from the group consisting of sorbitol, fructose, sorbitol 6-phosphate, myristate, palmitate and stearate. Multiple biomarkers may be correlated with CCPRCC, by any method, including statistical methods such as regression analysis.

After the level(s) of the one or more biomarker(s) is determined, the level(s) may be compared to CCPRCC reference level(s) or reference curves of the one or more biomarker(s) to determine a rating for each of the one or more biomarker(s) in the sample. The rating(s) may be aggregated using any algorithm to create a score, for example, a CCPRCC score, for the subject. The algorithm may take into account any factors relating to CCPRCC including the number of biomarkers, the correlation of the biomarkers to CCPRCC, etc.

In an embodiment, a mathematical model or formula containing one or more biomarkers as variables is established using regression analysis, e.g., multiple linear regressions. By way of non-limiting example, the developed formulas may include the following:

A+B(Biomarker₁)+C(Biomarker₂)+D(Biomarker₃)+E(Biomarker₄)=RScore

A+B*ln(Biomarker₁)+C*ln(Biomarker₂)+D*ln(Biomarker₃)+E*ln(Biomarker₄)=ln RScore,

wherein A, B, C, D, E are constant numbers;
Biomarker₁, Biomarker₂, Biomarker₃, Biomarker₄ are the measured values of the analyte (Biomarker) and RScore is the measure of cancer presence or absence or cancer aggressivity.

The formulas may include one or more biomarkers as variables, such as 1, 2, 3, 4, or more biomarkers.

Additionally, in one embodiment, the biomarkers provided herein to diagnose or aid in the diagnosis of CCPRCC may be used to distinguish CCPRCC from other urological cancers. A method of distinguishing CCPRCC from other urological cancers in a subject comprises (1) analyzing a biological sample from a subject to determine the level(s) of one or more biomarkers of CCPRCC in the sample and (2) comparing the level(s) of the one or more biomarkers in the sample to CCPRCC-positive and/or CCPRCC-negative reference levels of the one or more biomarkers in order to distinguish CCPRCC from other urological cancers. The one or more biomarkers that are used are selected from the group consisting of sorbitol, fructose, sorbitol 6-phosphate, myristate, palmitate and stearate. For example, one or more of the following biomarkers may be used alone or in any combination to distinguish CCPRCC from other urological cancers: sorbitol, fructose, sorbitol 6-phosphate, myristate, palmitate and stearate. When such a method is used to distinguish CCPRCC from other urological cancers, the results of the method may be used along with other methods (or the results thereof) useful in the clinical determination of distinguishing CCPRCC from other urological cancers.

The biomarkers and algorithms described herein may guide or assist a physician in deciding a treatment path, for example, whether to implement procedures such as surgical procedures (e.g., full or partial nephrectomy), treat with drug therapy, or employ a watchful waiting approach.

In one embodiment, the combination of biomarkers sorbitol, fructose, sorbitol 6-phosphate, myristate, palmitate, and stearate may provide a beneficial result.

In another embodiment, the combination of biomarkers sorbitol, fructose, sorbitol 6-phosphate, myristate, and palmitate may provide a beneficial result.

In another embodiment, the combination of biomarkers sorbitol, fructose, sorbitol 6-phosphate, myristate, and stearate may provide a beneficial result.

In another embodiment, the combination of biomarkers sorbitol, fructose, sorbitol 6-phosphate, palmitate, and stearate may provide a beneficial result.

In another embodiment, the combination of biomarkers sorbitol, fructose, myristate, palmitate, and stearate may provide a beneficial result.

In another embodiment, the combination of biomarkers sorbitol, sorbitol 6-phosphate, myristate, palmitate, and stearate may provide a beneficial result.

In another embodiment, the combination of biomarkers fructose, sorbitol 6-phosphate, myristate, palmitate, and stearate may provide a beneficial result.

In another embodiment, the combination of biomarkers fructose, sorbitol 6-phosphate, myristate, and palmitate may provide a beneficial result.

In another embodiment, the combination of biomarkers sorbitol, sorbitol 6-phosphate, myristate, and palmitate may provide a beneficial result.

In another embodiment, the combination of biomarkers sorbitol, fructose, myristate, and palmitate may provide a beneficial result.

In another embodiment, the combination of biomarkers sorbitol, fructose, sorbitol 6-phosphate, and palmitate may provide a beneficial result.

In another embodiment, the combination of biomarkers sorbitol, fructose, sorbitol 6-phosphate, and myristate may provide a beneficial result.

In another embodiment, the combination of biomarkers fructose, sorbitol 6-phosphate, myristate, and stearate may provide a beneficial result.

In another embodiment, the combination of biomarkers sorbitol, sorbitol 6-phosphate, myristate, and stearate may provide a beneficial result.

In another embodiment, the combination of biomarkers sorbitol, fructose, myristate, and stearate may provide a beneficial result.

In another embodiment, the combination of biomarkers sorbitol, fructose, sorbitol 6-phosphate, and stearate may provide a beneficial result.

In another embodiment, the combination of biomarkers fructose, sorbitol 6-phosphate, palmitate, and stearate may provide a beneficial result.

In another embodiment, the combination of biomarkers sorbitol, sorbitol 6-phosphate, palmitate, and stearate may provide a beneficial result.

In another embodiment, the combination of biomarkers sorbitol, fructose, palmitate, and stearate may provide a beneficial result.

In another embodiment, the combination of biomarkers fructose, myristate, palmitate, and stearate may provide a beneficial result.

In another embodiment, the combination of biomarkers sorbitol, myristate, palmitate, and stearate may provide a beneficial result.

In another embodiment, the combination of biomarkers sorbitol 6-phosphate, myristate, palmitate, and stearate may provide a beneficial result.

In another embodiment, the combination of biomarkers fructose, sorbitol 6-phosphate, and myristate may provide a beneficial result.

In another embodiment, the combination of biomarkers sorbitol, sorbitol 6-phosphate, and myristate may provide a beneficial result.

In another embodiment, the combination of biomarkers sorbitol, fructose, and myristate may provide a beneficial result.

In another embodiment, the combination of biomarkers sorbitol, fructose, and sorbitol 6-phosphate may provide a beneficial result.

In another embodiment, the combination of biomarkers fructose, sorbitol 6-phosphate, and stearate may provide a beneficial result.

In another embodiment, the combination of biomarkers sorbitol, sorbitol 6-phosphate, and stearate may provide a beneficial result.

In another embodiment, the combination of biomarkers sorbitol, fructose, and stearate may provide a beneficial result.

In another embodiment, the combination of biomarkers fructose, myristate, and stearate may provide a beneficial result.

In another embodiment, the combination of biomarkers sorbitol, myristate, and stearate may provide a beneficial result.

In another embodiment, the combination of biomarkers sorbitol 6-phosphate, myristate, and stearate may provide a beneficial result.

In another embodiment, the combination of biomarkers fructose, sorbitol 6-phosphate, and palmitate may provide a beneficial result.

In another embodiment, the combination of biomarkers sorbitol, sorbitol 6-phosphate, and palmitate may provide a beneficial result.

In another embodiment, the combination of biomarkers sorbitol, fructose, and palmitate may provide a beneficial result.

In another embodiment, the combination of biomarkers fructose, myristate, and palmitate may provide a beneficial result.

In another embodiment, the combination of biomarkers sorbitol, myristate, and palmitate may provide a beneficial result.

In another embodiment, the combination of biomarkers sorbitol 6-phosphate, myristate, and palmitate may provide a beneficial result.

In another embodiment, the combination of biomarkers fructose, palmitate, and stearate may provide a beneficial result.

In another embodiment, the combination of biomarkers sorbitol, palmitate, and stearate may provide a beneficial result.

In another embodiment, the combination of biomarkers sorbitol 6-phosphate, palmitate, and stearate may provide a beneficial result.

In another embodiment, the combination of biomarkers myristate, palmitate, and stearate may provide a beneficial result.

In another embodiment, the combination of biomarkers sorbitol, and stearate may provide a beneficial result.

In another embodiment, the combination of biomarkers sorbitol, and palmitate may provide a beneficial result.

In another embodiment, the combination of biomarkers sorbitol, and myristate may provide a beneficial result.

In another embodiment, the combination of biomarkers sorbitol, and sorbitol 6-phosphate may provide a beneficial result.

In another embodiment, the combination of biomarkers sorbitol, and fructose may provide a beneficial result.

In another embodiment, the combination of biomarkers fructose, and stearate may provide a beneficial result.

In another embodiment, the combination of biomarkers fructose, and palmitate may provide a beneficial result.

In another embodiment, the combination of biomarkers fructose, and myristate may provide a beneficial result.

In another embodiment, the combination of biomarkers fructose, and sorbitol 6-phosphate may provide a beneficial result.

In another embodiment, the combination of biomarkers sorbitol 6-phosphate, and stearate may provide a beneficial result.

In another embodiment, the combination of biomarkers sorbitol 6-phosphate, and palmitate may provide a beneficial result.

In another embodiment, the combination of biomarkers sorbitol 6-phosphate, and myristate may provide a beneficial result.

In another embodiment, the combination of biomarkers myristate, and stearate may provide a beneficial result.

In another embodiment, the combination of biomarkers myristate, and palmitate may provide a beneficial result.

In another embodiment, the combination of biomarkers palmitate, and stearate may provide a beneficial result.

In another embodiment, the biomarker sorbitol may provide a beneficial result.

In another embodiment, the biomarker fructose may provide a beneficial result.

In another embodiment, the biomarker sorbitol 6-phosphate may provide a beneficial result.

In another embodiment, the biomarker myristate may provide a beneficial result.

In another embodiment, the biomarker palmitate may provide a beneficial result.

In another embodiment, the biomarker stearate may provide a beneficial result.

### III. EXAMPLES

The invention will be further explained by the following illustrative examples that are intended to be non-limiting.

### I. General Methods

A. Identification of Metabolic Profiles for CCPRCC

Each sample was analyzed to determine the concentration of several hundred metabolites. Analytical techniques such as GC-MS (gas chromatography-mass spectrometry) and LC-MS (liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis

The data were plotted using scaled intensity of the given metabolite to identify molecules present at differential levels in a definable population or subpopulation (e.g., biomarkers for CCPRCC biological samples compared to ccRCC biological samples or compared to control biological samples) useful for distinguishing between the definable populations (e.g., CCPRCC, ccRCC and control). Other molecules in the definable population or subpopulation were also identified.

Principal Component Analysis (PCA) was performed to characterize the metabolic differences between CCPRCC, ccRCC and normal human tissue biopsy samples. For the PCA, each principal component is a linear combination of all metabolites. The coefficients for the first component are determined by those that maximize the variance. The coefficients for the second component are chosen to maximize the variance with the constraint that it is orthogonal to the first component.

C. Biomarker Identification

Various peaks identified in the analyses (e.g. GC-MS, LC-MS, LC-MS-MS), including those identified as statistically significant, were subjected to a mass spectrometry based chemical identification process.

### Example 1

### Biopsy Tissue Biomarkers for CCPRCC

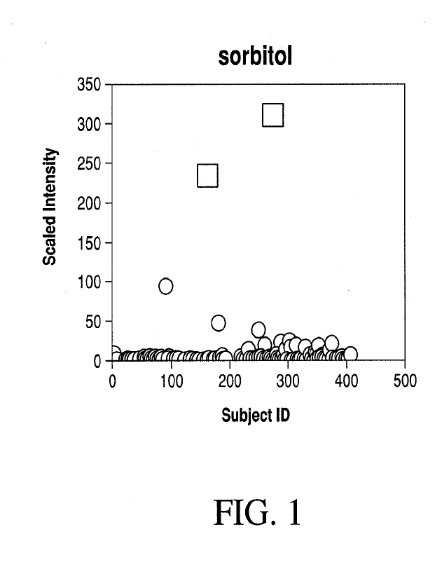

In a first study, 140 matched-pairs of tumor and normal kidney tissue were analyzed using metabolomics. In that study two cancer positive tumors had extremely high levels of sorbitol and were considered ‘outliers’. The data is presented in FIG. 1. Histological evaluation of those high sorbitol tumors revealed they were an RCC subtype that had been newly described as clear cell papillary RCC (CCPRCC). In a follow-up study this finding was confirmed. In the follow-up study biomarkers were identified by (1) analyzing tissue samples from human subjects to determine the levels of metabolites in the samples and then (2) analyzing the results to determine those metabolites that were differentially present in the kidney cancer tissue samples compared to the benign tissue samples.

The cohort was comprised of eleven CCPRCC-positive human kidney biopsies, 10 clear cell RCC positive and 10 patient-matched non-cancer human kidney biopsies. The cancer status of the sample was verified by histopathology analysis. Histological analysis was performed by a board-certified pathologist.

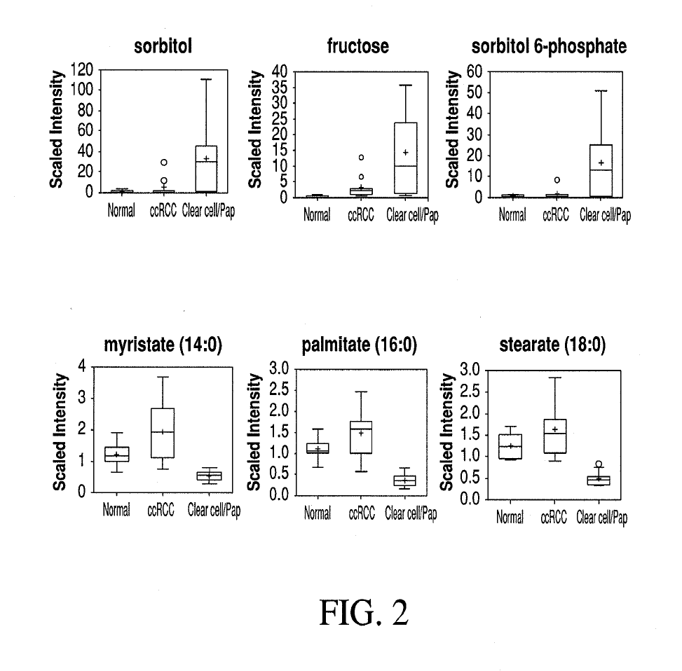

Metabolomic analysis of the samples resulted in the identification of 513 metabolites of known identity. After the levels of metabolites were determined, the data were plotted graphically to identify metabolites that were differentially altered in the CCPRCC samples compared to the clear cell RCC (ccRCC) and non-cancer samples. Levels of the biomarker metabolites sorbitol, fructose and sorbitol 6-phosphate are elevated in CCPRCC samples compared to normal and ccRCC samples. Levels of the biomarker metabolites myristate, palmitate and stearate are reduced in CCPRCC samples compared to normal and ccRCC samples. The box plots of the levels of the biomarker metabolites sorbitol, fructose, sorbitol 6-phosphate, myristate, palmitate and stearate are shown in FIG. 2.

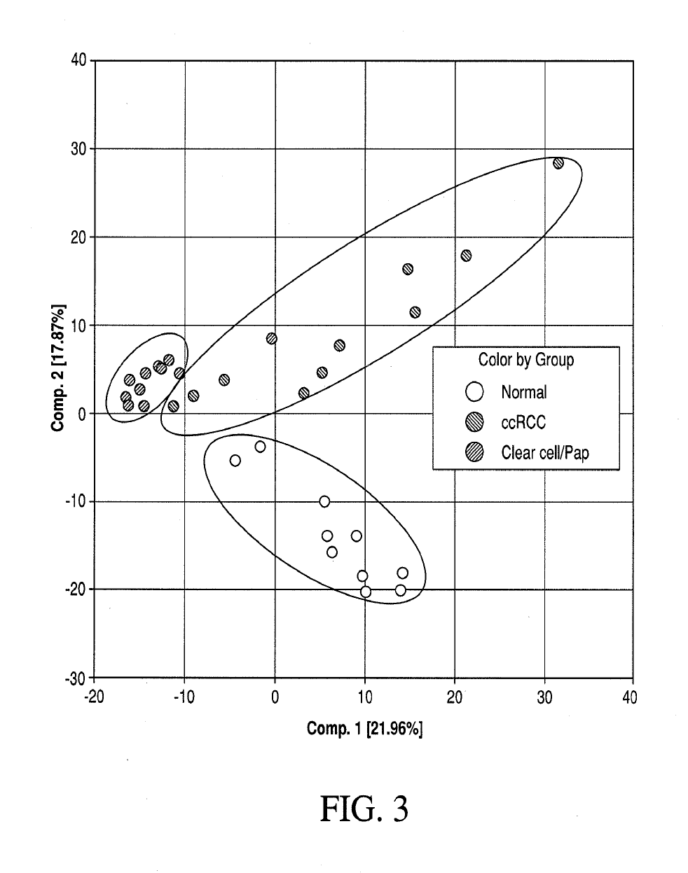

In addition, Principal Component Analysis (PCA) was carried out using all 513 of the biomarkers obtained from biopsy samples described above to classify the samples as CCPRCC, clear cell RCC or normal. A graphical depiction of the PCA results is presented in FIG. 3. From the graphic illustration of the PCA analysis, it can be seen that the samples from CCPRCC, ccRCC and normal patients are clustered together using only 2 biomarkers.

Further, using the mathematical model created using PCA, it was found that 10 of 11 CCPRCC samples were correctly classified as CCPRCC, 10 of 10 clear cell RCC samples were correctly classified, and 10 of 10 normal samples were correctly classified based on the biomarker abundance.

### Example 2

### Algorithm to Diagnose CCPRCC

Using the CCPRCC biomarkers, an algorithm could be developed to distinguish CCPRCC subjects. The algorithm, based on a panel of metabolite biomarkers from sorbitol, fructose, sorbitol 6-phosphate, myristate, palmitate and stearate, when used on a new set of patients, could assess the probability that the individual has CCPRCC. Using the results of this biomarker algorithm, a medical oncologist could assess the risk-benefit of surgery (i.e., full or partial nephrectomy), drug treatment or a watchful waiting approach.

The biomarker algorithm would monitor the levels of a panel of biomarkers for kidney cancer comprised of a plurality of biomarkers selected from the group consisting of sorbitol, fructose, sorbitol 6-phosphate, myristate, palmitate and stearate.

1. A method of diagnosing or aiding in diagnosing whether a subject has kidney cancer, comprising:
analyzing a biological sample from a subject to determine the level(s) of one or more biomarkers for kidney cancer in the sample, the group consisting of sorbitol, fructose, sorbitol 6-phosphate, myristate, palmitate, and stearate, and
comparing the level(s) of the one or more biomarkers in the sample to clear cell papillary kidney cancer-positive and/or kidney cancer-negative reference levels of the one or more biomarkers in order to diagnose whether the subject has clear cell papillary kidney cancer.

2. The method of claim 1, wherein the sample is analyzed using one or more techniques selected from the group consisting of mass spectrometry, ELISA, and antibody linkage.

3. The method of claim 2, wherein the method comprises analyzing the subject and a biological sample from the subject using a mathematical model comprising one or more biomarkers or measurements selected from the group consisting of sorbitol, fructose, sorbitol 6-phosphate, myristate, palmitate and stearate.

4. A method of distinguishing less aggressive kidney cancer from more aggressive kidney cancer in a subject having kidney cancer, comprising
analyzing a biological sample from a subject to determine the level(s) of one or more biomarkers for kidney cancer in the sample, wherein the one or more biomarkers are selected from the group consisting of sorbitol, fructose, sorbitol 6-phosphate, myristate, palmitate and stearate, and
comparing the level(s) of the one or more biomarkers in the sample to less aggressive kidney cancer and/or more aggressive kidney cancer reference levels of the one or more biomarkers in order to determine the aggressiveness of the subject's kidney cancer.

5. The method of claim 4, wherein a mathematical model is used to distinguish less aggressive kidney cancer from more aggressive kidney cancer in a subject having kidney cancer.

6. (canceled)

7. A method of distinguishing less aggressive kidney cancer from more aggressive kidney cancer in a subject having kidney cancer as described herein.
